(19) United States
(12) Patent Application Publication
(10) Pub. No.: US 2021/0380928 A1
(43) Pub. Date: Dec. 9, 2021

(54) CELL SEPARATION FILTER, FILTERING DEVICE, AND MANUFACTURING METHOD FOR CELL SEPARATION FILTER

(71) Applicant: FUJIFILM Corporation, Tokyo (JP)

(72) Inventors: Kazuomi INOUE, Ashigara-kami-gun (JP); Yosuke NAKAGAWA, Ashigara-kami-gun (JP); Kazuhide KANEMURA, Ashigara-kami-gun (JP); Ryuta TAKEGAMI, Ashigara-kami-gun (JP)

(73) Assignee: FUJIFILM Corporation, Tokyo (JP)

(21) Appl. No.: 17/408,140

(22) Filed: Aug. 20, 2021

Related U.S. Application Data
(63) Continuation of application No. PCT/JP2020/002214, filed on Jan. 23, 2020.

(30) Foreign Application Priority Data
Feb. 28, 2019 (JP) 2019-036128

(51) Int. Cl. C12N 1/02; B01D 29/00; D04H 1/728

(52) U.S. Cl. CPC C12N 1/02; B01D 29/0018; B01D 2239/0618; B01D 2257/91; D04H 1/728

(57) ABSTRACT

There are provide a cell separation filter with which cells can be separated without damage and can suppress adsorption, a filtering device, and a manufacturing method for a cell separation filter. The cell separation filter is composed of a nonwoven fabric that is formed of fibers containing a water-insoluble polymer and a hydrophilizing agent and has a fiber density difference in the film thickness direction. The nonwoven fabric has an average through-hole diameter of 2.0 μm or more and less than 10.0 μm, a void ratio of 75% or more and 98% or less, a film thickness of 100 μm or more, and a critical wet surface tension of 72 mN/m or more.

### CROSS-REFERENCE TO RELATED APPLICATIONS

This application is a Continuation of PCT International Application No. PCT/JP2020/002214 filed on Jan. 23, 2020, which claims priority under 35 U.S.C. § 119(a) to Japanese Patent Application No. 2019-036128 filed on Feb. 28, 2019. Each of the above applications is hereby expressly incorporated by reference, in its entirety, into the present application.

### BACKGROUND OF THE INVENTION

### 1. Field of the Invention

The present invention relates to a cell separation filter that is used for cell separation, a filtering device, and a manufacturing method for a cell separation filter and in particular, relates to a cell separation filter composed of a nonwoven fabric that is formed of fibers containing a water-insoluble polymer and a hydrophilizing agent and has a fiber density difference in the film thickness direction, a filtering device, and a manufacturing method for a cell separation filter.

### 2. Description of the Related Art

Currently, a nonwoven fabric composed of so-called nanofibers having a fiber diameter of 1 μm or less is expected to be used for various intended purposes. Nonwoven fabrics composed of nanofibers are used, for example, in filters for filtering liquids, and are proposed in, for example, JP2012-46843A, WO2018/101156, and JP1997-143081A (JP-H9-143081A).

JP2012-46843A discloses a filter medium containing a water-resistant cellulose sheet consisting of a nonwoven fabric composed of fine cellulose fibers having a number-average fiber diameter of 500 nm or less. The water-resistant cellulose sheet satisfies all of a weight ratio of fine cellulose fibers: 1% by mass or more and 99% by mass or less, a void ratio: 50% or more, a tensile strength equivalent to 10 g/m² weight: 6N/15 mm or more, and a wet and dry strength ratio of the tensile strength: 50% or more.

In addition, WO2018/101156 discloses a filtering medium for selective adsorption of blood components, as a substance for selectively removing blood components such as leukocytes, where the filtering medium contains cellulose acylate, has a glass transition temperature of 126° C. or higher, has an average through-hole diameter of 0.1 to 50 μm, and has a specific surface area of 1.0 to 100 m²/g. The form of the filtering medium for selective adsorption of blood components is a nonwoven fabric.

Further, JP1997-143081A (JP-H9-143081A) discloses a plasma separation filter with which a container having an inlet and an outlet is filled so that the average hydraulic radius of aggregates of ultrafine fibers composed of a nonwoven fabric is 0.5 μm to 3.0 μm and the ratio (L/D) between a flow path diameter (D) of a blood component and a flow path length (L) of blood is 0.15 to 6. The ultrafine fibers of JP1997-143081A (JP-H9-143081A) are polyester, polypropylene, polyamide, or polyethylene.

### SUMMARY OF THE INVENTION

A nonwoven fabric composed of nanofibers has a network structure formed by nanofibers. In a case where the nonwoven fabric is used as a filtering medium for a liquid, a filtration target such as a liquid passes through voids due to the network structure and is filtered.

However, in a case of being used for cell separation, there is a possibility that cells are not capable of being separated without damage with the filters of JP2012-46843A, WO2018/101156, JP1997-143081A (JP-H9-143081A), which are described above. In this case, there is a possibility that hemolysis occurs in a case where plasma is separated from the blood. Further, in the case of the use for cell separation, it is required that an object to be passed is not adsorbed to the filtering medium since the separation accuracy is decreased in a case where the object to be passed is adsorbed. This point is also not taken into consideration in JP2012-46843A, WO2018/101156, and JP1997-143081A (JP-119-143081A).

An object of the present invention is to provide a cell separation filter with which cells can be separated without damage and can suppress adsorption, a filtering device, and a manufacturing method for a cell separation filter.

For achieving the above object, the present invention provides a cell separation filter composed of a nonwoven fabric that is formed of fibers containing a water-insoluble polymer and a hydrophilizing agent and has a fiber density difference in the film thickness direction, where the nonwoven fabric has an average through-hole diameter of 2.0 μm or more and less than 10.0 μm, a void ratio of 75% or more and 98% or less, a film thickness of 100 μm or more, and a critical wet surface tension of 72 mN/m or more.

The hydrophilizing agent is preferably at least one of polyvinylpyrrolidone, polyethylene glycol, carboxymethyl cellulose, or hydroxypropyl cellulose.

The nonwoven fabric preferably has a film thickness of 200 μm or more and 2,000 μm or less.

The critical wet surface tension is preferably 85 mN/m or more.

The water-insoluble polymer is preferably any one of polyethylene, polypropylene, polyester, polysulfone, polyethersulfone, polycarbonate, polystyrene, a cellulose derivative, an ethylene vinyl alcohol polymer, polyvinyl chloride, polylactic acid, polyurethane, polyphenylene sulfide, polyamide, polyimide, polyvinylidene fluoride, polytetrafluoroethylene, or an acrylic resin, or a mixture thereof.

The water-insoluble polymer preferably consists of a cellulose derivative.

The content of the hydrophilizing agent with respect to the total mass of the fibers of the nonwoven fabric is preferably 1% to 50% by mass.

It is preferable that the fiber density of the nonwoven fabric continuously changes in the film thickness direction.

The present invention provides a filtering device that has the above-described cell separation filter according to an aspect of the present invention and in which the cell separation filter is arranged so that a filtration target passes through the cell separation filter from a low fiber density side to a high fiber density side in the film thickness direction.

The present invention provides a filtering device which has the above-described cell separation filter according to an aspect of the present invention and a porous body having an average through-hole diameter of 0.2 μm or more and 1.5 μm or less and having a void ratio of 60% or more and 95% or less, where the cell separation filter and the porous body are arranged so that a filtration target passes through the cell separation filter and the porous body in this order.

It is preferable that the cell separation filter is arranged so that a filtration target passes through the cell separation filter from a low fiber density side to a high fiber density side in the film thickness direction.

The present invention provides a manufacturing method for the above-described cell separation filter according to an aspect of the present invention, and in the manufacturing method for a cell separation filter, the cell separation filter is manufactured by using an electrospinning method.

According to the present invention, it is possible to obtain a cell separation filter with which cells can be separated without damage and adsorption can be suppressed and a filtering device.

In addition, it is possible to manufacture a cell separation filter with which cells can be separated without damage and can adsorption can be suppressed.

### BRIEF DESCRIPTION OF THE DRAWINGS

FIG. 1 is a schematic view illustrating an example of a cell separation filter according to the embodiment of the present invention.

FIG. 2 is a schematic cross-sectional view showing an example of the cell separation filter according to the embodiment of the present invention.

FIG. 3 is a graph showing an example of measurement results of the cell separation filter according to the embodiment of the present invention.

FIG. 4 is a graph showing the anisotropy of the cell separation filter according to the embodiment of the present invention.

FIG. 5 is a schematic view illustrating another example of the cell separation filter according to the embodiment of the present invention.

FIG. 6 is a schematic cross-sectional view showing an example of a conventional nonwoven fabric.

FIG. 7 is a graph showing an example of measurement results of a conventional nonwoven fabric.

FIG. 8 is a schematic view illustrating a first example of a filtering device according to the embodiment of the present invention.

FIG. 9 is a schematic view illustrating a second example of the filtering device according to the embodiment of the present invention.

FIG. 10 is a schematic view illustrating a third example of the filtering device according to the embodiment of the present invention.

FIG. 11 is a schematic view illustrating a fourth example of the filtering device according to the embodiment of the present invention.

FIG. 12 is a schematic view illustrating an example of a filtration system having a filtering device according to the embodiment of the present invention.

### DESCRIPTION OF THE PREFERRED EMBODIMENTS

Hereinafter, a cell separation filter, a filtering device, and a manufacturing method for a cell separation filter, according to the embodiment of the present invention, will be described in detail based on the suitable embodiments shown in the attached drawings.

It is noted that the figures explained below are exemplary for explaining the present invention, and the present invention is not limited to the figures shown below.

In the following, “to” indicating a numerical range includes numerical values described on both sides thereof. For example, in a case where s is a numerical value α to a numerical value β, the range of ε is a range including the numerical value α and the numerical value β and thus α≤ε≤β in a case of describing with mathematical symbols.

The “angle represented by a specific numerical value” and the “temperature represented by a specific numerical value” include an error range generally allowed in the related technical field unless otherwise specified.

(Cell Separation Filter)

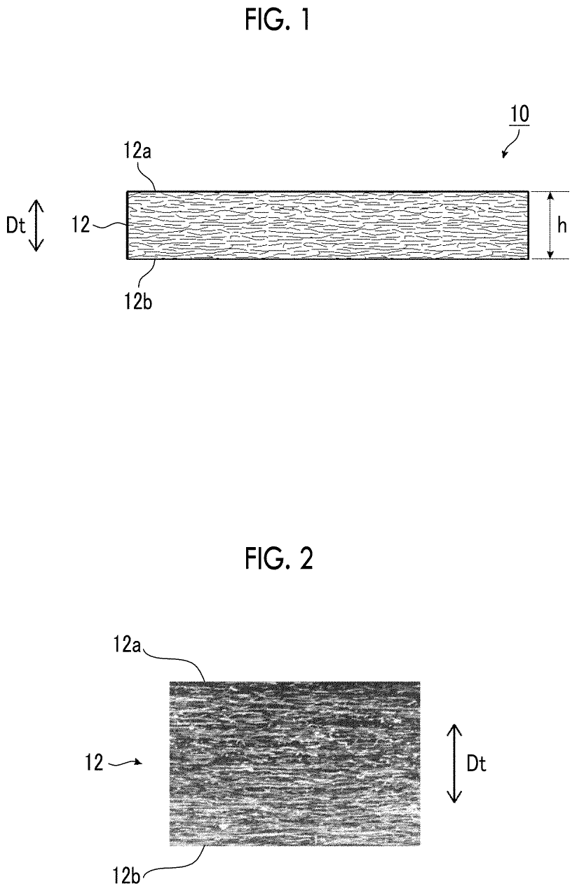

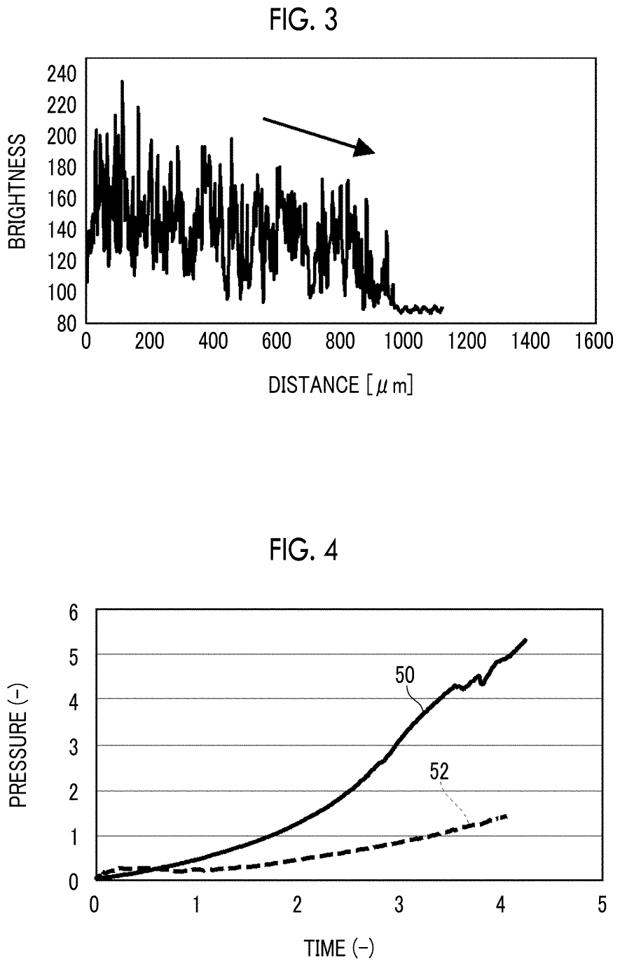

FIG. 1 is a schematic view illustrating an example of a cell separation filter according to the embodiment of the present invention, and FIG. 2 is a schematic cross-sectional view showing an example of the cell separation filter according to the embodiment of the present invention. FIG. 3 is a graph showing an example of measurement results of the cell separation filter according to the embodiment of the present invention.

A cell separation filter 10 illustrated in FIG. 1 is composed of a nonwoven fabric that is formed of fibers containing a water-insoluble polymer and a hydrophilizing agent and has a fiber density difference in the film thickness direction.

In the cell separation filter 10, the fiber density is different in the film thickness direction Dt as shown in FIG. 2. In FIG. 2, the fiber density on a back surface 12b side of the nonwoven fabric 12 is low, and the fiber density on a front surface 12a side is high.

The nonwoven fabric 12 constituting the cell separation filter 10 has an average through-hole diameter of 2.0 μm or more and less than 10.0 μm, a void ratio of 75% or more and 98% or less, and a film thickness h (see FIG. 1) of 100 μm or more, and a critical wet surface tension of 72 mN/m or more.

With the above configuration, the cell separation filter 10 can separate cells without damaging cells and can suppress adsorption. The separation with the cell separation filter 10 includes filtering elimination as well as filtration. The separation target of the cell separation filter 10 is not particularly limited as long as it contains cells, and is blood or the like.

For example, in a case where the separation target is blood, the cell separation filter 10 can suppress hemolysis during plasma separation.

In a case where blood is filtered through the cell separation filter 10, leukocytes, erythrocytes, and blood cell components of platelets can be removed, and thus it is possible to obtain plasma proteins, sugars, lipids, electrolytes, and the like, which are necessary for the examination, with being left in the plasma. As described above, it is possible to suppress the adsorption of plasma proteins, sugars, lipids, electrolytes, and the like, which are necessary for the examination. As a result, the examination accuracy can be improved.

Regarding the cell separation filter 10, the separation target, the size that can be filtered, and the like are collectively referred to as separation characteristics.

In the present invention, the filtration target is not limited to blood, and body fluids such as lymph, saliva, urine, and tear fluid are also filtration targets in addition to blood. Further, in the present invention, cells derived from animals such as human cells, cells derived from plants, cells derived from microorganisms, and the like can be subjected to filtering elimination. Examples of the above-described cells include somatic stem cells such as hematopoietic stem cells, myeloid stem cells, nerve stem cells, and skin stem cells, embryonic stem cells, induced pluripotent stem cells, and cancer cells.

In addition, the following cells are also separation targets of the present invention; leukocytes such as neutrophils, eosinophils, basophils, monocytes, and lymphocytes (T cells, natural killer (NK) cells, B cells, and the like), blood cells such as platelets, erythrocytes, vascular endothelial cells, lymphoid stem cells, erythroblasts, myeloblasts, monoblasts, megakaryoblasts and megakaryocytes, endothelial cells, epithelial cells, hepatic parenchymal cells, and pancreatic islet of langerhans cells, as well as various established cell line cells for research.

In the cell separation filter 10, instead of the filtration target, a filtering elimination target can also be supplied and subjected to filtering elimination.

Hereinafter, the cell separation filter will be described more specifically.

<Nonwoven Fabric>

As described above, the cell separation filter is composed of a nonwoven fabric formed of fibers containing a water-insoluble polymer and a hydrophilizing agent.

The nonwoven fabric preferably consists of fibers having an average fiber diameter of 1 nm or more and 5 μm or less and having an average fiber length of 1 mm or more and 1 m or less, more preferably consists of nanofibers having an average fiber diameter of 100 nm or more and less than 1,000 nm and having an average fiber length of 1.5 mm or more and 1 m or less, and still more preferably consists of nanofibers having an average fiber diameter of 100 nm or more and 800 nm or less and having an average fiber length of 2.0 mm or more and 1 m or less.

The average fiber diameter and the average fiber length can be adjusted, for example, by adjusting the concentration of a solution at the time of manufacturing the nonwoven fabric.

Here, the average fiber diameter refers to a value measured as follows.

A transmission electron microscope image or a scanning electron microscope image of the surface of a nonwoven fabric consisting of fibers is obtained.

The electron microscope image is obtained at a magnification selected from 1,000 to 5,000 times depending on the size of the fibers constituting the nonwoven fabric. However, the sample, the observation conditions, and magnification are adjusted so that the following conditions are satisfied.

(1) A straight line X is drawn at any position in the electron microscope image so that 20 or more fibers intersect the straight line X.

(2) In the same electron microscope image, a straight line Y that perpendicularly intersects the straight line X is drawn so that 20 or more fibers intersect the straight line Y.

Regarding each of the fibers crossing the straight line X and the fibers crossing the straight line Y in the electron microscope image as described above, the width (the short diameter of the fiber) of at least 20 fibers (that is, at least 40 fibers in total) is read. In this manner, at least 3 sets or more of the above-described electron microscope images are observed, and fiber diameters of at least 40 fibers×3 sets (that is, at least 120 fibers) are read.

The average fiber diameter is obtained by averaging the fiber diameters read in this manner.

In addition, the average fiber length refers to a value measured as follows.

That is, the fiber length of the fiber can be obtained by analyzing the electron microscope image that is used in measuring the above-described average fiber diameter.

Specifically, regarding each of the fibers crossing the straight line X and the fibers crossing the straight line Y in the electron microscope image as described above, the fiber length of at least 20 fibers (that is, at least 40 fibers in total) is read.

In this manner, at least 3 sets or more of the above-described electron microscope images are observed, and fiber lengths of at least 40 fibers×3 sets (that is, at least 120 fibers) are read.

The average fiber length is obtained by averaging the fiber lengths read in this manner.

<Fiber Density Difference>

Regarding the fiber density difference in the film thickness direction of the nonwoven fabric constituting the cell separation filter, in a case where the fiber density difference is small, cake filtration occurs, and the processing pressure increases. On the other hand, in a case where the fiber density difference is large, stepwise filtration is possible, and the processing pressure can be decreased. In a case where the processing pressure is high and blood is filtered, erythrocyte destruction easily occurs, which leads to an increase in the degree of hemolysis.

The processing pressure corresponds to the pressure loss during the filtration. The low processing pressure means that the resistance of the cell separation filter during the filtration is low. In a case where the processing pressure is low, the pressure required for filtration can be decreased.

The pressure loss is the difference between a static pressure on the front surface side and a static pressure on the back surface side in the film thickness direction across the cell separation filter. Accordingly, the pressure loss can be determined by measuring the static pressure on the front surface side and the static pressure on the back surface side and obtaining the difference between the two static pressures. The pressure loss can be measured using a differential pressure gauge.

Here, the fiber density correlates with the brightness of the X-ray computed tomography (CT) image, and the fiber density can be specified by the brightness. For example, the result shown in FIG. 3 can be obtained. The higher the brightness of the X-ray CT image, the higher the fiber density. In FIG. 3, as the distance value increases, the brightness tends to decrease, and thus the fiber density decreases.

The fiber density difference in the film thickness direction is obtained by carrying out a cross-sectional X-ray CT image analysis in the film thickness direction. First, a cross-sectional X-ray CT image is acquired, the entire film thickness in the cross-sectional X-ray CT image is equally divided into 10 sections in the film thickness direction, and the brightness in each of the sections is integrated. The integrated brightnesses are denoted by L1, L2, L3, L4, L5, L6, L7, L8, L9, and L10 in order from the lowest brightness.

“There is a fiber density difference in the film thickness direction” refers to that the ratio of the minimum value of brightness to the maximum value of brightness is L1/L10<0.95. In this case, it is preferable that, among one surface and the other surface, the fiber density of any one surface is maximal, and the fiber density of the remaining surface is minimal. That is, it is preferable that, among the front surface 12a and the back surface 12b of the nonwoven fabric 12, the fiber density of any one surface is maximal, and the fiber density of the remaining surface is minimal.

As shown in FIG. 4, in a case where a fiber density difference is present in the film thickness direction, the pressure required for filtration is different in the film thickness direction between a case where filtration is carried out from a surface with the higher fiber density (see a pressure curve 50) and a case where filtration is carried out from a surface with the lower fiber density (see a pressure curve 52). That is, the cell separation filter 10 has anisotropy in the film thickness direction. In a case where a filtration target is allowed to pass from a low fiber density side to a high fiber density side in the film thickness direction, it is possible to reduce the pressure required for filtration.

In addition, FIG. 4 shows the results of carrying out filtration using the same liquid and changing only the direction of the cell separation filter 10. Both the pressure and the time in FIG. 4 are both dimensionless.

In the cell separation filter, the pressure required for filtration can be reduced due to the fiber density difference in the film thickness direction, whereby cells can be separated without damage, and for example, blood can be filtered with hemolysis being suppressed.

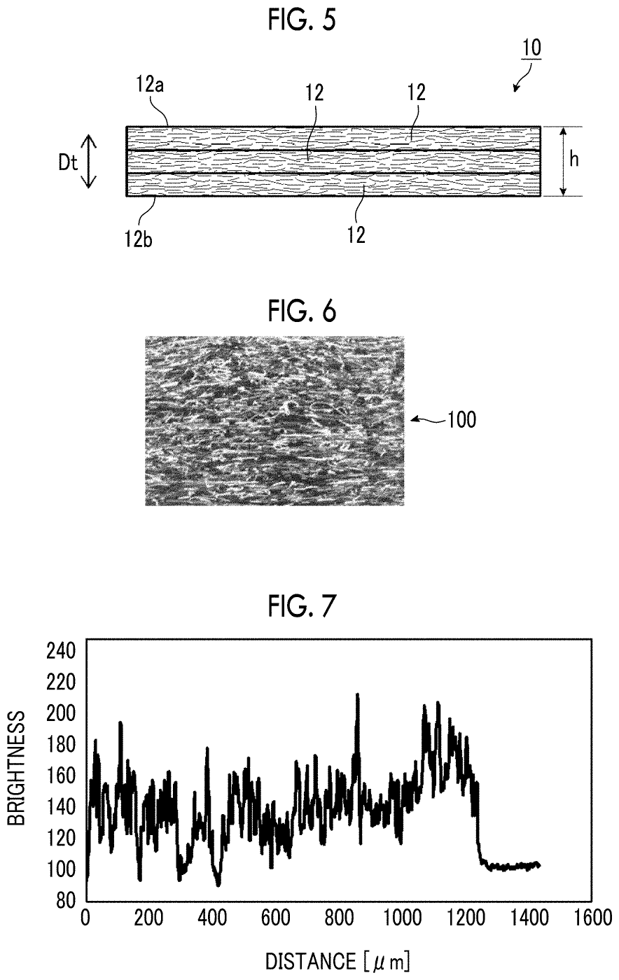

The cell separation filter 10 is not limited to being composed of one nonwoven fabric, that is, a single layer, and may have a configuration in which a plurality of nonwoven fabrics 12 are laminated as in the cell separation filter 10 illustrated in FIG. 5. In this case, the cell separation filter 10 has an interface in the film thickness direction Dt, and thus in the cell separation filter 10, the fiber density discontinuously changes, which will be described later.

Here, FIG. 6 is a schematic cross-sectional view showing an example of a conventional nonwoven fabric, and FIG. 7 is a graph showing an example of measurement results of a conventional nonwoven fabric.

As shown in FIG. 6, in a conventional nonwoven fabric 100, the fibers are not unevenly distributed. Further, the fiber density is not biased as shown in the graph of the brightness of the X-ray CT image in FIG. 7. In the conventional nonwoven fabric, there is no fiber density difference in the film thickness direction and the fiber density is not different in the specific direction, and thus the conventional nonwoven fabric is isotropic. As a result, even in a case where the supply direction of a filtration target is changed, there is no big difference in the pressure required for filtration.

“The fiber density continuously changes in the film thickness direction” refers to that the above-described brightnesses L1 to L10 satisfy 0.9<Ln/Ln+1<1.05. Here, n is 1 to 9.

In a case where the fiber density continuously changes in the film thickness direction, it is referred to that the fiber density has a gradient in the film thickness direction.

In a case where the fiber density continuously changes in the film thickness direction, it is preferable that there is no sudden change in the fiber density. However, it is permissible that the fiber density reversely varies in a part of the 10 sections which are obtained by being equally divided into 10 sections in the film thickness direction described above. That is, in a case where L1/L10<0.95 is satisfied, the fiber density is not limited to being that the fiber density represented by the brightness gradually increases or gradually decreases in one direction in the above-described 10 sections which are obtained by being equally divided into 10 sections in the film thickness direction, and sections having the same fiber density may be adjacent to each other.

The above-described L1/L10 is more preferably 0.3<L1/L10<0.95, still more preferably 0.4<L1/L10<0.9, and most preferably 0.5<L1/L10<0.9.

<Average Through-Hole Diameter>

The average through-hole diameter is preferably 2.0 μm or more and less than 10.0 μm, more preferably 2.0 μm or more and less than 8.0 μm, still more preferably 3.0 μm or more and less than 7.0 μm, and most preferably 3.0 μm or more and less than 5.0 μm.

In a case where the average through-hole diameter is small with respect to the size of the filtration target, the processing pressure is high, and in a case where it is large with respect to the size of the filtration target, the processing pressure is low.

The degree of hemolysis is a degree of erythrocyte destruction. Accordingly, in a case where the average through-hole diameter is smaller than the size of erythrocytes, the erythrocytes are crushed on the cell separation filter, and thus the degree of hemolysis increases, resulting in poor filter performance.

In a case where the average through-hole diameter is large, erythrocytes pass easily, and in a case where a secondary filter is present, the cells are crushed by the secondary filter, and thus the degree of hemolysis increases. In a case where the average through-hole diameter is large and there is no secondary filter, erythrocytes get mixed and the component matching rate after filtration decreases. In this case as well, the performance of the filter is decreased.

As described above, the degree of hemolysis is a degree of erythrocyte destruction. The degree of hemolysis can be calculated by (the amount of hemoglobin in the plasma (the filtrate))/(the amount of hemoglobin in the whole blood). In general, erythrocytes are destroyed by the pressure due to osmotic pressure or pressure due to physical compression, chemical actions such as electrostatic interaction, or biological actions such as complement activation, and hemoglobin is released and colored red. The degree of hemolysis can be determined by measuring hemoglobin in the plasma by spectrometric measurement.

The average through-hole diameter can be measured with a palm porometer by using a bubble point method (Japanese Industrial Standards (JIS) K3832, ASTM F316-86)/Half-dry Method (ASTM E1294-89). Hereinafter, the average through-hole diameter will be described in detail.

Regarding the “average through-hole diameter”, in a pore diameter distribution measurement test using a palm porometer (CFE-1200AEX, manufactured by Seika Corporation), the air pressure is increased by 2 cc/min with respect to a sample completely wetted with GALWICK (Porous Materials, Inc.) in the same manner as in the method described in paragraph <0093> of JP2012-046843A, and evaluation is carried out. Specifically, with respect to a film-shaped sample completely wetted with GALWICK (1,1,2,3,3,3-hexafluoropropene; manufactured by Porous Materials, Inc.), a predetermined amount of air is sent at 2 cc/min to one side of the film, and while measuring the pressure, the flow rate of the air permeating to the opposite side of the film is measured. From this method, first, data on the pressure and the permeating air flow rate (hereinafter, also referred to as “wet curve”) is obtained for the film-shaped sample which has been wetted with GALWICK. Next, the same data (hereinafter, also referred to as “dry curve”) is measured for a non-wet, dry film-shaped sample, and a pressure at an intersection of a curve (a half dry curve) corresponding to half of the flow rate of the dry curve and the wet curve) are obtained. Thereafter, the surface tension (γ) of GALWICK, the contact angle (θ) with the filtering medium, and the air pressure (P) are introduced into the following Expression (I) to calculate the average through-hole diameter.

Average through-hole diameter=4γ cos θ/P (I)

Examples of the method for adjusting the average through-hole diameter include the methods described below.

((Control of Fiber Diameter))

In the method for controlling the fiber diameter, which is one of the methods for adjusting the average through-hole diameter, the fiber diameter can be controlled by changing the solvent, the concentration of the material, the voltage, and the like, which are used at the time of spinning by electrospinning. Since there is a proportional relationship between the fiber diameter and the average through-hole diameter, the average through-hole diameter can be adjusted by controlling the fiber diameter.

((Heat Fusion Welding))

In the method using heat fusion welding, which is one of the methods for adjusting the average through-hole diameter, the fibers can be fusion-welded to each other and the average through-hole diameter can be reduced. In heat fusion welding, unlike the control of the fiber diameter, the average through-hole diameter can only be reduced.

((Calender Treatment))

In the method using calender treatment, which is one of the methods for adjusting the average through-hole diameter, the average through-hole diameter can be reduced by pressurizing and crushing fibers with a roller or the like to firmly sticking the fibers. In calender treatment, unlike the control of the fiber diameter, the average through-hole diameter can only be reduced.

<Void Ratio>

The void ratio is preferably 75% or more and 98% or less, more preferably 85% or more and 98% or less, and still more preferably 90% or more and 98% or less.

The higher the void ratio is, the more hardly the cake filtration occurs, and the more hardly the processing pressure increases. As a result, the supply speed of the filtration target can be increased at the time of filtration. On the other hand, in a case where the void ratio is low, it is easy to shift to the cake filtration, and the processing pressure tends to increase.

The void ratio is calculated as follows.

First, in a case where the void ratio is denoted by Pr (%), the film thickness of a nonwoven fabric of a square of 10 cm×10 cm is denoted by Hd (μm), and the mass of a nonwoven fabric of a square of 10 cm×10 cm is denoted by Wd (g), the void ratio is calculated using Pr=(Hd−Wd×67.14)×100/Hd.

<Film Thickness>

In the cell separation filter, the nonwoven fabric has a film thickness h (see FIG. 1) of 100 μm or more, and the film thickness is preferably 200 μm or more and 2,000 μm or less and more preferably 200 μm or more and 1,000 μm or less.

The film thickness h of the nonwoven fabric (see FIG. 1) is the film thickness of the cell separation filter.

In a case where the film thickness is not equal to or more than a predetermined thickness, there is no fiber density difference. In a case where the film thickness is too thin, the components desired to be removed cannot be completely removed, which leads to a decrease in the component matching rate.

On the other hand, in a case where the film thickness is too thick, high pressure is required to cause all the separation targets such as the filtration target to permeate, and thus the processing pressure tends to be high and the degree of hemolysis tends to be high. Further, in a case where the film thickness is too thick, the volume of contact with the biological component increases, which leads to a decrease in the component matching rate.

For the film thickness, a cross-sectional observation of the nonwoven fabric is carried out using a scanning electron microscope to obtain a cross-sectional image. Using the cross-sectional image, 10 points as the film thickness of the nonwoven fabric were measured, and the average value thereof was taken as the film thickness.

<Critical Wet Surface Tension>

Critical wet surface tension (CWST) is a parameter representing wettability.

The critical wet surface tension (CWST) is 72 millinewtons per meter (mN/m) or more, and the critical wet surface tension (CWST) is preferably 85 mN/m or more.

In a case where the critical wet surface tension (CWST) is high, the filtration target such as blood easily spreads wettably on the nonwoven fabric, and thus the effective area becomes large and the blood processing pressure tends to decrease.

In a case where the critical wet surface tension (CWST) is low, the effective area becomes small and the blood processing pressure tends to increase. Further, in a case where the critical wet surface tension (CWST) is low, biological substances are easily adsorbed, which leads to a decrease in the component matching rate. Critical wet surface tension (CWST) can be controlled with the amount of the hydrophilizing agent or the alkali treatment.

The definition of critical wet surface tension (CWST) is as follows.

The critical wet surface tension can be determined by observing the absorption or non-absorption of each liquid on the surface while changing the surface tension of the liquid, which is applied onto the measurement target surface, by 2 mN/m to 4 mN/m.

The unit of CWST is mN/m, which is defined as the average value of the surface tension of the absorbed liquid and the surface tension of the adjacent unabsorbed liquid. As an example, the surface tension of the absorbed liquid is 27.5 mN/m, and the surface tension of the unabsorbed liquid is 52 mN/m. In a case where a surface tension interval is an odd number, for example, 3, then it can be determined whether the nonwoven fabric is closer to the lower value or closer to the higher value, and based on this determination, 27 or 28 is assigned to the nonwoven fabric.

In measuring CWST, a series of standard test liquids of which the surface tension sequentially changes by about 2 to about 4 mN/m are prepared. Each liquid of at least two standard liquids of which the surface tensions are sequential, where each liquid has a diameter of 3 to 5 mm, is placed on a nonwoven fabric, left for 10 minutes, and observed after 10 to 11 minutes. The case of being “wet” is defined in a case where at least 9 out of 10 liquid droplet are absorbed by the nonwoven fabric, that is wetted, within 10 minutes.

Being non-wet is defined by non-wetting, that is, non-absorption of two or more liquid droplets within 10 minutes. Using continuous high or low surface tension liquids, the test is continued until one of the pair with the narrowest surface tension is determined as wet and the other is determined as non-wet.

CWST is within the range of these conditions, and for convenience, the average of the two surface tensions can be used as one number to specify the CWST. In a case where the two test liquids differ by 3 mN/m, it is determined which test liquids the test piece is closer to and an integer is assigned as described above. Solutions with different surface tensions can be made by various methods. Specific examples are shown below.

Sodium hydroxide aqueous solutions: 94 to 115 (mN/m)

Calcium chloride aqueous solutions: 90 to 94 (mN/m)

Sodium nitrate aqueous solutions: 75 to 87 (mN/m)

Pure water: 72.4 (mN/m)

Acetic acid aqueous solutions 38 to 69 (mN/m)

Ethanol aqueous solutions: 22 to 35 (mN/m)

<Water-Insoluble Polymer>

The water-insoluble polymer is a polymer having a solubility of less than 0.1% by mass in pure water.

As a specific example, the water-insoluble polymer is preferably any one of polyethylene, polypropylene, polyester, polysulfone, polyethersulfone, polycarbonate, polystyrene, a cellulose derivative, an ethylene vinyl alcohol polymer, polyvinyl chloride, polylactic acid, polyurethane, polyphenylene sulfide, polyamide, polyimide, polyvinylidene fluoride, polytetrafluoroethylene, or an acrylic resin, or a mixture thereof. Since the cellulose derivative has smaller adsorption of biological substances than other materials, the component matching rate is good. Accordingly, the water-insoluble polymer is more preferably a cellulose derivative.

The cellulose derivative refers to a modified cellulose obtained by chemically modifying a part of hydroxy groups contained in cellulose which is a natural polymer. The chemical modification of the hydroxy group is not particularly limited, and examples thereof include the alkyl etherification of the hydroxy group, the hydroxyalkyl etherification, and the esterification. The cellulose derivative has at least one hydroxy group in one molecule. Only one kind of cellulose derivative may be used, or two or more kinds thereof may be used in combination.

Examples of the cellulose derivative include methyl cellulose, ethyl cellulose, propyl cellulose, butyl cellulose, hydroxyethyl cellulose, hydroxypropyl cellulose, hydroxypropyl methyl cellulose, hydroxybutyl methyl cellulose, cellulose acetate (acetyl cellulose, diacetyl cellulose, triacetyl cellulose, and the like), cellulose acetate propionate, cellulose acetate butyrate, and nitrocellulose.

Further, in the fibers constituting the nonwoven fabric, the content of the water-insoluble polymer is preferably 50% to 99% by mass, more preferably 70% to 93% by mass, and still more preferably 85% to 93% by mass, with respect to the total mass of the fibers of the nonwoven fabric.

In a case where the content of the water-insoluble polymer is less than 50% by mass, the strength of the fibers forming the nonwoven fabric is decreased, the shape is easily changed by filtration, whereby the processing pressure is increased. On the other hand, in a case where the content of the water-insoluble polymer is 99% by mass or more, the amount of the hydrophilizing agent is reduced, and the hydrophilization effect of the fibers forming the nonwoven fabric is reduced. From this reason, the content of the water-insoluble polymer is preferably 50% to 99% by mass.

<Hydrophilizing Agent>

The hydrophilizing agent is a material having a solubility of 1% by mass or more in pure water.

As a specific example, the hydrophilizing agent is preferably at least one of polyvinylpyrrolidone, polyethylene glycol, carboxymethyl cellulose, or hydroxypropyl cellulose, and the hydrophilizing agent is most preferably polyvinylpyrrolidone.

Further, in the fibers constituting the nonwoven fabric, the content of the hydrophilizing agent is preferably 1% to 50% by mass, more preferably 5% to 30% by mass, and still more preferably 7% to 15% by mass, with respect to the total mass of the fibers of the nonwoven fabric.

In a case where the content of the hydrophilizing agent is more than 50% by mass, the strength of the fibers forming the nonwoven fabric is decreased, the shape is easily changed by filtration, whereby the processing pressure is increased. On the other hand, in a case where the content of the hydrophilizing agent is less than 1% by mass, the amount of the hydrophilizing agent is small, and the hydrophilization effect of the fibers forming the nonwoven fabric is reduced. From this reason, the content of the hydrophilizing agent is preferably 1% to 50% by mass.

(Manufacturing Method for Cell Separation Filter)

As described above, the cell separation filter is composed of a nonwoven fabric that is formed of fibers containing a water-insoluble polymer and a hydrophilizing agent and has a fiber density difference in the film thickness direction.

The cell separation filter is manufactured using an electric field spinning method, also called an electrospinning method. With this method, it is possible to manufacture a cell separation filter with which cells can be separated without damage and can adsorption can be suppressed.

A manufacturing method using an electrospinning method will be described. First, for example, a solution in which the above-described water-insoluble polymer and the hydrophilizing agent are dissolved in a solvent is discharged from the distal end of a nozzle at a predetermined temperature within a range of 5° C. or higher and 40° C. or lower, a voltage is applied between the solution and a collector to eject fibers from the solution onto a support provided on the collector, and then the nanofibers are collected, whereby a nanofiber layer, that is, a nonwoven fabric can be obtained. In this case, the voltage applied between the solution and the collector is adjusted to change the fiber density at the time of ejecting fibers. The fiber density can also be changed by adjusting the concentration of the solution.

As the manufacturing device, for example, the nanofiber manufacturing device disclosed in JP6132820B can be used. The solution contains a water-insoluble polymer and a hydrophilizing agent dissolved in a solution, and the water-insoluble polymer and the hydrophilizing agent are not separately injected from a nozzle to be spun.

Since the cell separation filter is not limited to a single layer as described above, nonwoven fabrics having different fiber densities may be prepared by the electrospinning method as described above, and these may be laminated to manufacture the cell separation filter, in the order of fiber density from the nonwoven fabric having a lower fiber density.

(Filtering Device)

It is possible to constitute a filtering device using the cell separation filter described above. The filtering device, like the cell separation filter, can separate cells without damaging cells and can suppress adsorption.

The filtering device has a cell separation filter, and the cell separation filter is arranged so that a filtration target passes through the cell separation filter from a low fiber density side to a high fiber density side in the film thickness direction. In a case where the cell separation filter is arranged so that a filtration target is allowed to pass from a low fiber density side to a high fiber density side in the film thickness direction, it is possible to reduce the processing pressure. As a result, the pressure required for filtration can be reduced.

The filtering device may have a configuration having, in addition to the cell separation filter, a porous body in which an average through-hole diameter is 0.2 μm or more and 1.5 μm or less and a void ratio is 60% or more and 95% or less. In this case, the cell separation filter and the porous body are arranged so that a filtration target passes through the cell separation filter and the porous body in this order.

Hereinafter, the filtering device will be specifically described.

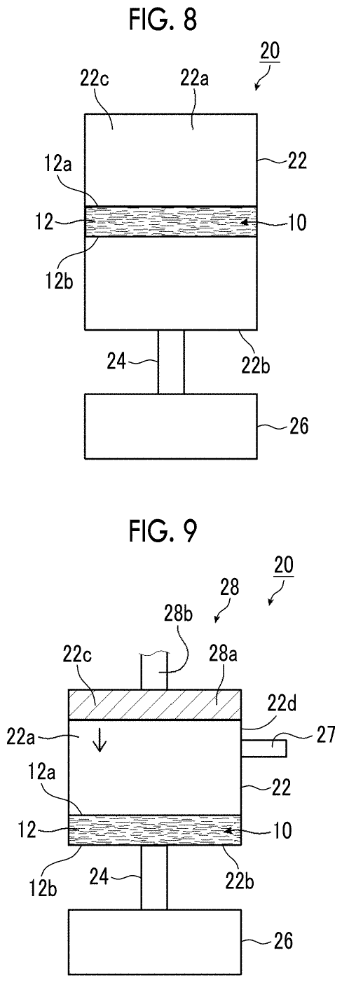

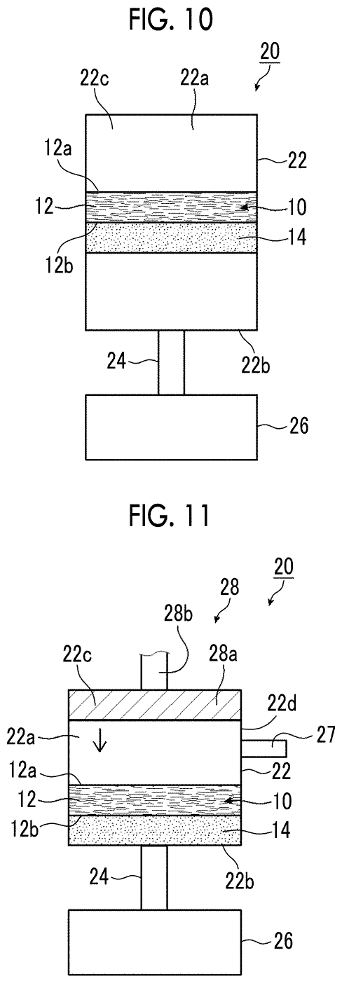

FIG. 8 is a schematic view illustrating a first example of the filtering device according to the embodiment of the present invention, and FIG. 9 is a schematic view illustrating a second example of the filtering device according to the embodiment of the present invention. FIG. 10 is a schematic view illustrating a third example of the filtering device according to the embodiment of the present invention, and FIG. 11 is a schematic view illustrating a fourth example of the filtering device according to the embodiment of the present invention.

In the filtering devices of FIG. 8 to FIG. 11, the same components as those of the cell separation filter 10 illustrated in FIG. 1 are designated by the same references, and detailed descriptions thereof will be omitted.

In a filtering device 20 illustrated in FIG. 8, for example, a disk-shaped cell separation filter 10 is provided in an inside 22a of a cylindrical case 22. In a bottom part 22b of the case 22, a connecting pipe 24 is provided at the center of the bottom part 22b. The connecting pipe 24 is connected to a collection unit 26.

In the case 22, an end on a side opposite to bottom part 22b is opened. The portion that is opened is called an opening portion 22c. A filtration target is supplied from the opening portion 22c, filtered by the cell separation filter, passed through the connecting pipe 24 from the bottom part 22b of the case 22, and the filtered filtration target is stored in a collection unit 26.

In the filtering device 20, instead of the filtration target, a filtering elimination target can also be supplied and subjected to filtering elimination. In this case, a filtering elimination target is supplied from the opening portion 22c, subjected to filtering elimination by the cell separation filter, passed through the connecting pipe 24 from the bottom part 22b of the case 22, and the filtering elimination target undergone filtering elimination is stored in a collection unit 26.

Further, as illustrated in FIG. 9, the filtering device 20 may have a configuration having a pressurizing part 28. The pressurizing part 28 is provided in the opening portion 22c of the case 22. The pressurizing part 28 has a gasket 28a provided in the opening portion 22c and arranged without a gap between the gasket and the inside 22a of the case 22 and a plunger 28b which moves the gasket 28a in the direction from the opening portion 22c toward the bottom part 22b or in the opposite direction. In a case where the plunger 28b is moved toward the bottom part 22b, the filtration target in the inside 22a of the case 22 can be allowed to permeate through the cell separation filter 10 to be filtered.

In a case where the pressurizing part 28 is provided, a supply pipe 27 communicating with the inside 22a of the case 22 may be provided on the outer surface 22d of the case 22. The supply pipe 27 is provided closer to the opening portion 22c than to the cell separation filter 10.

Further, in the filtering device 20 having the pressurizing part 28, instead of the filtration target, a filtering elimination target can also be supplied and subjected to filtering elimination.

Further, as illustrated in FIG. 10, the filtering device 20 may have a configuration having an object having a filter function in addition to the cell separation filter 10. The object having a filter function preferably an object having separation characteristics different from those of the cell separation filter 10. In this case, even those that cannot be completely filtered by the cell separation filter 10 can be filtered, and thus the separation accuracy can be improved.

The filtering device 20 illustrated in FIG. 10 is different from the filtering device 20 illustrated in FIG. 8 in that a porous body 14 is provided on the bottom part 22b side of the case 22 of the cell separation filter 10, and the configuration other than the above is the same as that of the filtering device 20 illustrated in FIG. 8.

For example, the porous body 14 is provided to be in contact with the back surface 12b of the nonwoven fabric 12 constituting the cell separation filter 10. The filtration target is supplied from the cell separation filter 10 side. In the filtering device 20 illustrated in FIG. 10, the cell separation filter 10 is also referred to as a primary filter, and the porous body 14 is also referred to as a secondary filter.

The porous body 14 has an average through-hole diameter of 0.2 μm or more and 1.5 μm or less and has a void ratio of 60% or more and 95% or less. The separation characteristics thereof are different from those of the cell separation filter 10.

The porous body 14 can be composed of, for example, the same material as that of the nonwoven fabric 12 and can be composed of fibers containing the water-insoluble polymer and the hydrophilizing agent that constitute the nonwoven fabric 12. Since the definition of the average through-hole diameter and the void ratio of the porous body 14 is the same as that of the cell separation filter 10, detailed descriptions thereof will be omitted.

In the filtering device 20 illustrated in FIG. 10, the cell separation filter 10 and the porous body 14 are provided, and thus even those that cannot be completely filtered by the cell separation filter 10 can be filtered, whereby the separation accuracy can be improved.

In the filtering device 20 illustrated in FIG. 10, for example, in a case of filtering blood, erythrocytes and leukocytes are removed by the cell separation filter 10, and platelets are removed by the porous body 14. As a result, plasma proteins, sugars, lipids, electrolytes, and the like, which necessary for the examination, can be obtained, and hemolysis can be further suppressed.

The filtering device 20 illustrated in FIG. 10 can also have a configuration in which the pressurizing part 28 is provided in the same manner as in the filtering device 20 illustrated in FIG. 9. Since the pressurizing part 28 has the same configuration as the filtering device 20 illustrated in FIG. 9, detailed descriptions thereof will be omitted. Further, the supply pipe 27 may be provided in the same manner as in the filtering device 20 illustrated in FIG. 9.

In addition, the porous body 14 is not limited to the above-described configurations, and those matching with the separation characteristics of the cell separation filter 10, the filtration target, or the filtering elimination target can be appropriately used. However, it is preferable that the separation characteristics are different from those of the cell separation filter 10 as described above.

Further, in the above, although one porous body 14 is provided in addition to the cell separation filter 10, the configuration is not limited to this, and a plurality of objects having a filter function, like the porous body 14, may be provided.

The cell separation filter 10 and the porous body 14 are not limited to be provided to be in contact with each other, and the cell separation filter 10 and the porous body 14 may be arranged to be spaced apart from each other in the film thickness direction of the cell separation filter 10.

Any one of the above-described filtering devices 20 has a configuration in which one cell separation filter 10 is provided; however, the configuration is not limited to this, and a plurality of cell separation filters 10 may be provided.

Further, in any one of the above-described filtering devices 20, the position of the cell separation filter 10 is not particularly limited as long as it is in the inside 22a of the case 22, and may be spaced apart from the bottom part 22b of the case 22, or may be in contact with the bottom part 22b of the case 22. The cell separation filter 10 may be installed in the case 22 in a state where the nonwoven fabric is provided in a flat film shape in a housing (not shown) with respect to the case 22.

Further, in any one of the above-described filtering devices 20, the collection unit 26 may not be provided, and the bottom part 22b may be closed without the connecting pipe 24 and the collection unit 26 being provided. In a case where the bottom part 22b is closed, the filtered material may be allowed to be stored in the bottom part 22b.

Further, in a case where the bottom part 22b is closed, an opening that communicates with the inside 22a of the case 22 may be provided at the bottom part 22b so that the filtered material is taken out to the outside.

(Filtration System)

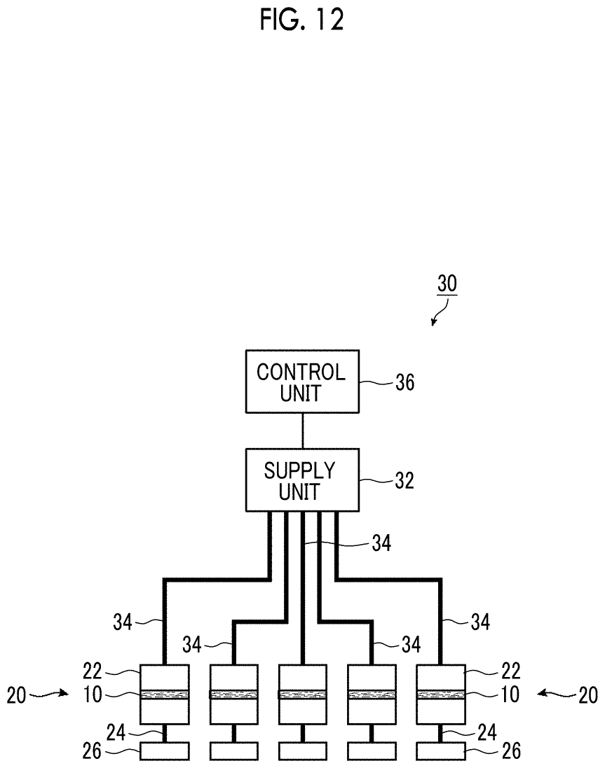

It is noted that any one of the above-described filtering devices 20 is not limited to being used alone. Here, FIG. 12 is a schematic view illustrating an example of a filtration system having a filtering device according to the embodiment of the present invention.

As in a filtration system 30 illustrated in FIG. 12, a configuration in which a plurality of filtering devices 20 are provided, and each of the filtering devices 20 is allowed to automatically filter a filtration target may be adopted.

In FIG. 12, the same configuration components as those of the filtering device 20 illustrated in FIG. 8 are designated by the same references, and the detailed description thereof will be omitted.

The filtration system 30 illustrated in FIG. 12 includes a supply unit 32, a plurality of filtering devices 20 that are connected to the supply unit 32 by a pipe 34, and a control unit 36 that controls the supply unit 32.

The supply unit 32 supplies a filtration target to each of the filtering devices 20 and has a storage unit (not illustrated in the figure) for storing a filtration target and a pump (not illustrated in the figure) for supplying the filtration target from the storage unit to the filtering device 20. As the pump, for example, a syringe pump is used. The pump such as a syringe pump is controlled by a control unit 36, and the filtration target is supplied from the storage unit to the filtering device 20 by the pump, filtered, and collected at the collection unit 26.

In the filtration system 30, the filtering device 20 may also have a pressurizing part 28 as illustrated in FIG. 9. In this case, a driving unit (not illustrated in the figure) for moving the plunger 28b of the pressurizing part 28 is provided. In a case where the driving unit and the pump are controlled by the control unit 36, filtration can be automatically executed as described above.

Since the processing pressure for the cell separation filter 10 can be reduced, the pressure required for filtration can be reduced and the time required for filtration can be shortened in the filtration system 30. As a result, the power consumption of the filtration system 30 can be reduced.

In the filtration system 30, instead of the filtration target, a filtering elimination target can also be supplied and subjected to filtering elimination.

The present invention is basically configured as described above. As described above, the cell separation filter, the filtering device, and the manufacturing method for a cell separation filter, according to the embodiment of the present invention, have been described in detail; however, the present invention is not limited to the above-described embodiments, and, of course, various improvements or modifications may be made without departing from the gist of the present invention.

### EXAMPLES

Hereinafter, the characteristics of the present invention will be described more specifically with reference to Examples. The materials, reagents, amounts of substances and their ratios, operations, and the like in the following Examples can be appropriately changed as long as they do not depart from the gist of the present invention. Accordingly, the scope of the present invention is not limited to the following Examples.

In present examples, cell separation filters of Examples 1 to 20 and Comparative Examples 1 to 7 were prepared. The following blood filtration tests were carried out using each of the cell separation filters to evaluate the degree of hemolysis, the processing pressure, and the component matching rate after filtration.

[Evaluation]

In the blood filtration test, whole blood is diluted with a buffer, blood cell components (leukocytes, erythrocytes, and platelets) are removed by filtration, and it is aimed to test whether plasma proteins, sugars, lipids, electrolytes, and the like, which are necessary for the examination, are obtained without loss, that is, left in the plasma. Hereinafter, the blood filtration test will be described.

First, the cell separation filter was cut out to a diameter of 25 mm and set in a filter holder (SWINNEX, manufactured by Merck Millipore) together with an O-ring. 5 ml of fresh human whole blood (anticoagulant: EDTA-2K) was mixed with 25 mL of a buffer and allowed to flow in a direction perpendicular to the cell separation filter so that the low density side of the cell separation filter was the primary side, that is, the side where the liquid was supplied, and filtered.

(Degree of Hemolysis)

Regarding the degree of hemolysis, the degree of hemolysis of the filtered plasma was measured using HemoCue (registered trade mark) manufactured by Radiometer.

A degree of hemolysis of less than 1% was evaluated as A, a degree of hemolysis of 1% or more and less than 4% was evaluated as B, a degree of hemolysis of 4% or more and less than 10% was evaluated as C, and a degree of hemolysis of 10% or more was evaluated as D.

(Processing Pressure)

The pressure loss during the filtration was measured, and the pressure loss was used as the processing pressure. A pressure loss of less than 20 kPa was evaluated as A, a pressure loss of 20 kPa or more and less than 40 kPa was evaluated as B, a pressure loss of 40 kPa or more and less than 80 kPa was evaluated as C, and a pressure loss of 80 kPa or more was evaluated as D. The pressure loss was measured using a differential pressure gauge. A small digital pressure gauge GC31 (trade name) manufactured by NAGANO KEIKI Co., Ltd. was used as the differential pressure gauge.

(Component Matching Rate)

Regarding the component matching rate, the amount of albumin in the plasma obtained by centrifuging the whole blood before filtration and the amount of albumin in the plasma after filtration were measured. The component matching rate was calculated by comparing the amount of albumin in each of the plasmas. The amount of albumin was measured using an albumin measurement kit (product code: DIAG-250) manufactured by Funakoshi Co., Ltd.

A component matching rate of 98% or more was evaluated as A, a component matching rate of less than 98% and 95% or more was evaluated as B, a component matching rate of less than 95% and 90% or more was evaluated as C, and a component matching rate of less than 90% was evaluated as D.

[Characteristics of Cell Separation Filter]

(Average Through-Hole Diameter)

The average through-hole diameter was measured with a palm porometer by using a bubble point method (Japanese Industrial Standards (JIS) K3832, ASTM F316-86)/Half-dry Method (ASTM E1294-89).

(Void Ratio)

Regarding the void ratio, as described above, in a case where the void ratio was denoted by Pr (%), the film thickness of a nonwoven fabric of a square of 10 cm×10 cm was denoted by Hd (μm), and the mass of a nonwoven fabric of a square of 10 cm×10 cm was denoted by Wd (g), the void ratio was calculated using Pr=(Hd−Wd×67.14)×100/Hd.

(Film Thickness)

For the film thickness, a cross-sectional observation of the nonwoven fabric is carried out using a scanning electron microscope to obtain a cross-sectional image. Using the cross-sectional image, 10 points as the film thickness of the nonwoven fabric were measured, and the average value thereof was taken as the film thickness.

(Critical Wet Surface Tension (CWST))

The critical wet surface tension (CWST), which represents wettability, was controlled by the amount of the hydrophilizing agent or the alkali treatment. The method for measuring the critical wet surface tension (CWST) is described below.

Solutions having different surface tensions are prepared. 10 drops of 10 μL of the solution are gently placed on a horizontally leveled cell separation filter and left for 10 minutes. In a case where 9 or more drops out of 10 are wet, it is determined that the cell separation filter is wetted by the solution of its surface tension. In a case of being wetted, a solution having a surface tension higher than that of the wetted solution used and is dropped in the same manner, and the procedure is repeated until 2 or more drops out of the 10 drops are no longer wetted. In a case where 2 or more drops out of the 10 drops are not wetted, it is determined that the cell separation filter is not wetted by the solution of its surface tension, and the average value of the surface tensions of the wetted solution and the non-wetted solution is defined as the critical wet surface tension (CWST) of the cell separation filter.

The difference in the surface tension between the wetted solution and the non-wetted solution is set to be within 2 mN/m, and the measurement is carried out in a standard laboratory atmosphere (Japanese Industrial Standards (JIS) K7100) at a temperature of 23° C. and a relative humidity of 50%. For measurements at different temperatures or humidity, in a case where there exists a conversion table, the table is used to calculate the wetting tension. The criterion for determining that the dropped solution is wet is that the contact angle between the cell separation filter and the solution is 90° or less.

Acetic acid aqueous solutions (54 to 70 mN/m) and sodium hydroxide aqueous solutions (72 to 100 mN/m) were used for the critical wet surface tension (CWST) measurement, and the surface tension of the prepared solution was measured with an automatic surface tension meter (manufactured by Kyowa Interface Science Co., Ltd., Wilhelmy plate method) under the same conditions as the environment in which the critical wet surface tension (CWST) was measured.

(Fiber Density Difference)

For the fiber density difference, an X-ray computed tomography (CT) image in the film thickness direction of the cell separation filter is acquired, and the entire film thickness in the cross-sectional X-ray CT image is equally divided into 10 sections in the film thickness direction. The brightness in each of the sections which are obtained by being equally divided into 10 sections was integrated. The integrated brightnesses were denoted by L1, L2, L3, L4, L5, L6, L7, L8, L9, and L10 in order from the lowest brightness, a value of L1/L10 was determined, and this value was used as the fiber density difference.

Further, it was checked whether or not 0.9<Ln/Ln+1<1.05 was satisfied for the above-described brightnesses L1 to L10. In a case where 0.9<Ln/Ln+1<1.05 was satisfied, it was described as “Continuous” in the fiber density gradient column, and in a case where the above was not satisfied, it was described as “Discontinuous” in the fiber density gradient column.

In Table 1 to Table 4 below, the materials described in the alphabetical notation are materials shown below.

CAP: Cellulose acetate propionate

TAC: Triacetyl cellulose

DAC: Diacetyl cellulose

PP: Polypropylene

PET: Polyethylene terephthalate

PSU: Polysulfone

CMC: Carboxymethyl cellulose

PVP: Polyvinylpyrrolidone

HPC: Hydroxypropyl cellulose

The average through-hole diameter, the void ratio, the film thickness, the critical wet surface tension (CWST), the fiber density difference, the fiber density gradient, the material, and the manufacturing method of Examples 1 to 20 and Comparative Examples 1 to 7 are shown in Table 1 to Table 4 below.

Hereinafter, Examples 1 to 20 and Comparative Examples 1 to 7 will be described.

### Example 1

In Example 1, a nonwoven fabric was manufactured by an electrospinning method using cellulose acetate propionate (CAP) as a water-insoluble polymer and polyvinylpyrrolidone (PVP) as a hydrophilizing agent, and used as a cell separation filter. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

For the nonwoven fabric using the electrospinning method, the nanofiber manufacturing device disclosed in JP6132820A was used, the temperature of the spinning solution coming out of the nozzle was set to 20° C., the flow rate of the spinning solution coming out of the nozzle was set to 20 mL/hour, and the voltage applied between the solution and the collector was adjusted in a range of 10 to 40 kV, and the nanofibers were collected on a support made of an aluminum sheet having a thickness of 25 μm, which was arranged on the collector, whereby a nonwoven fabric was obtained.

The above-described water-insoluble polymer and hydrophilizing agent were dissolved in a mixed solvent of 80% by mass of dichloromethane and 20% by mass of methanol so that the total solid content concentration was 10% by mass, and used as a spinning solution. The ratios of the water-insoluble polymer and the hydrophilizing agent described in Example 1, Examples 2 to 20, and Comparative Examples 1 to 7 shown below is the details of the above-described solid content. This is the same as the ratio of the water-insoluble polymer and the hydrophilizing agent to the total mass of the fibers of the nonwoven fabric.

The fact that cellulose acetate propionate (CAP) is 90% by mass of the total solid content in the mixed solvent is indicated as “CAP/90%” in the “Material” column of Table 1. The fact that polyvinylpyrrolidone (PVP) is 10% by mass of the total solid content in the mixed solvent is indicated as “PVP/10%” in the “Hydrophilizing agent” column of Table 1.

In the following description, in Example 1, it is simply referred to that cellulose acetate propionate (CAP) is 90% by mass and polyvinylpyrrolidone (PVP) is 10% by mass. Hereinafter, substances other than the above will be indicated in the same manner as in Example 1.

In Example 1, the average through-hole diameter is 3.1 μm, the void ratio is 97%, the critical wet surface tension is 85 mN/m, the film thickness is 800 μm, and the fiber density difference is 0.85, and the fiber density gradient is continuous.

### Example 2

In Example 2, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Example 2, a nonwoven fabric was manufactured by an electrospinning method in the same manner as in Example 1 except that the average through-hole diameter and the fiber density difference were changed as shown in Table 1 described later, and used as the cell separation filter. Cellulose acetate propionate (CAP) is 90% by mass, and polyvinylpyrrolidone (PVP) is 10% by mass. In Example 2, the average through-hole diameter is 5.0 μm, and the fiber density difference is 0.88 as compared with Example 1.

### Example 3

In Example 3, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Example 3, a nonwoven fabric having a film thickness of 400 μm was formed by the electrospinning method in the same manner as in Example 1 except that the average through-hole diameter and the fiber density difference were changed as shown in Table 1 described later and the fiber density gradient was made discontinuous, and then the manufacturing was once stopped and the surface of the nonwoven fabric was statically eliminated with a static eliminator (manufactured by MILTY, a static electricity removal pistol Zerostat 3 (trade name)). Subsequently, the surface of the statically eliminated nonwoven fabric was subjected to the spinning again by the electrospinning method under the same conditions so that the total film thickness was 800 μm. In this manner, a nonwoven fabric having a discontinuous fiber density was manufactured and used as the cell separation filter. Cellulose acetate propionate (CAP) is 90% by mass, and polyvinylpyrrolidone (PVP) is 10% by mass. In Example 3, the average through-hole diameter is 5.0 μm, and the fiber density difference is 0.88 as compared with Example 1.

### Example 4

In Example 4, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Example 4, three nonwoven fabrics were manufactured by an electrospinning method in the same manner as in Example 1 except that the average through-hole diameter, the film thickness, and the fiber density difference were changed as shown in Table 1 described later, and the three nonwoven fabrics were laminated and used as the cell separation filter. Cellulose acetate propionate (CAP) is 90% by mass, and polyvinylpyrrolidone (PVP) is 10% by mass. In Example 4, the fiber density gradient of one nonwoven fabric is continuous, but the fiber density gradient is discontinuous as a cell separation filter. In Example 4, the average through-hole diameter is 5.0 μm, the film thickness is 250 μm, and the fiber density difference is 0.93 as compared with Example 1.

### Example 5

In Example 5, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Example 5, a nonwoven fabric was manufactured by an electrospinning method in the same manner as in Example 1 except that the average through-hole diameter and the fiber density difference were changed as shown in Table 1 described later, and used as the cell separation filter. Cellulose acetate propionate (CAP) is 90% by mass, and polyvinylpyrrolidone (PVP) is 10% by mass. In Example 5, the average through-hole diameter is 2.1 μm, and the fiber density difference is 0.88 as compared with Example 1.

### Example 6

In Example 6, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Example 6, a nonwoven fabric was manufactured by an electrospinning method in the same manner as in Example 1 except that the average through-hole diameter and the fiber density difference were changed as shown in Table 1 described later, and used as the cell separation filter. Cellulose acetate propionate (CAP) is 90% by mass, and polyvinylpyrrolidone (PVP) is 10% by mass. In addition, the film thickness is 800 μm. In Example 6, the average through-hole diameter is 9.7 μm, and the fiber density difference is 0.87 as compared with Example 1.

### Example 7

Example 7 is the same as Example 6 except that a porous body composed of polysulfone (PSU) is arranged below the cell separation filter as compared with Example 6. The porous body was prepared by the method disclosed in Example 2 of JP1987-27006A (JP-S62-27006A). The porous body has an average through-hole diameter of 0.8 μm, a void ratio of 85%, and a thickness of 150 μm. In Example 7, the average through-hole diameter is 9.7 μm, and the fiber density difference is 0.87 as compared with Example 1.

The porous body was prepared by the method disclosed in Example 2 of JP1987-27006A (JP-S62-27006A). Further, as the polysulfone (PSU), Udel (registered trade mark) P-3500 LCD MB manufactured by Solvay S.A. was used.

### Example 8

In Example 8, triacetyl cellulose (TAC) was used as a water-insoluble polymer, and polyvinylpyrrolidone (PVP) was used as a hydrophilizing agent. For triacetyl cellulose (TAC), M-300 (trade name) manufactured by Eastman Chemical Company, Japan was used, and for polyvinylpyrrolidone (PVP), K-90 manufactured by Nippon Shokubai Co., Ltd. was used.

In Example 8, a nonwoven fabric was manufactured by an electrospinning method in the same manner as in Example 1 except that the average through-hole diameter, the void ratio, the film thickness, and the fiber density difference were changed as shown in Table 2 described later, and used as the cell separation filter. Triacetyl cellulose (TAC) is 90% by mass, and polyvinylpyrrolidone (PVP) is 10% by mass. In Example 8, the average through-hole diameter is 4.4 μm, the void ratio is 96%, the film thickness is 500 μm, and the fiber density difference is 0.90 as compared with Example 1.

### Example 9

In Example 9, diacetyl cellulose (DAC) was used as a water-insoluble polymer, and polyvinylpyrrolidone (PVP) was used as a hydrophilizing agent. For diacetyl cellulose (DAC), CA-320S (trade name) manufactured by Eastman Chemical Company, Japan was used, and for polyvinylpyrrolidone (PVP), K-90 manufactured by Nippon Shokubai Co., Ltd. was used.

In Example 9, a nonwoven fabric was manufactured by an electrospinning method in the same manner as in Example 1 except that the average through-hole diameter, the void ratio, the film thickness, and the fiber density difference were changed as shown in Table 2 described later, and used as the cell separation filter. Diacetyl cellulose (DAC) is 90% by mass, and polyvinylpyrrolidone (PVP) is 10% by mass. In Example 9, the average through-hole diameter is 4.1 μm, the void ratio is 96%, the film thickness is 500 μm, and the fiber density difference is 0.84 as compared with Example 1.

### Example 10

In Example 10, triacetyl cellulose (TAC) was used as a water-insoluble polymer, and polyvinylpyrrolidone (PVP) was used as a hydrophilizing agent. For triacetyl cellulose (TAC), M-300 (trade name) manufactured by Eastman Chemical Company, Japan was used, and for polyvinylpyrrolidone (PVP), K-90 manufactured by Nippon Shokubai Co., Ltd. was used.

In Example 10, a nonwoven fabric was manufactured by an electrospinning method in the same manner as in Example 1 except that the average through-hole diameter, the void ratio, the film thickness, and the fiber density difference were changed as shown in Table 2 described later, and used as the cell separation filter. Triacetyl cellulose (TAC) is 90% by mass, and polyvinylpyrrolidone (PVP) is 10% by mass. In Example 10, the average through-hole diameter is 3.8 μm, the void ratio is 98%, the film thickness is 150 μm, and the fiber density difference is 0.94 as compared with Example 1.

### Example 11

In Example 11, triacetyl cellulose (TAC) was used as a water-insoluble polymer, and polyvinylpyrrolidone (PVP) was used as a hydrophilizing agent. For triacetyl cellulose (TAC), M-300 (trade name) manufactured by Eastman Chemical Company, Japan was used, and for polyvinylpyrrolidone (PVP), K-90 manufactured by Nippon Shokubai Co., Ltd. was used.

In Example 11, a nonwoven fabric was manufactured by an electrospinning method in the same manner as in Example 1 except that the average through-hole diameter, the void ratio, the film thickness, and the fiber density difference were changed as shown in Table 2 described later, and used as the cell separation filter. Triacetyl cellulose (TAC) is 90% by mass, and polyvinylpyrrolidone (PVP) is 10% by mass. Further, in Example 11, the average through-hole diameter is 7.2 μm, the void ratio is 95%, the film thickness is 200 μm, and the fiber density difference is 0.94 as compared with Example 1.

### Example 12

In Example 12, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Example 12, a nonwoven fabric was manufactured by an electrospinning method in the same manner as in Example 1 except that the average through-hole diameter, the critical wet surface tension, and the fiber density difference were changed as shown in Table 2 described later, and used as the cell separation filter. Cellulose acetate propionate (CAP) is 92.5% by mass, and polyvinylpyrrolidone (PVP) is 7.5% by mass. In Example 12, the amount of polyvinylpyrrolidone (PVP) is reduced to reduce the critical wet surface tension, the critical wet surface tension is 72 mN/m, the average through-hole diameter is 3.3 μm, and the fiber density difference is 0.90 as compared with Example 1.

### Example 13

In Example 13, triacetyl cellulose (TAC) was used as a water-insoluble polymer, and hydroxypropyl cellulose (HPC) was used as a hydrophilizing agent. For triacetyl cellulose (TAC), M-300 (trade name) manufactured by Eastman Chemical Company, Japan was used, and for hydroxypropyl cellulose (HPC), product number 088-03865 (viscosity: 0.15 to 0.4 Pa·s (150 to 400 cP)) manufactured by FUJIFILM Wako Pure Chemical Corporation was used.

In Example 13, a nonwoven fabric was manufactured by an electrospinning method in the same manner as in Example 1 except that the average through-hole diameter, the critical wet surface tension, and the fiber density difference were changed as shown in Table 2 described later, and used as the cell separation filter. Triacetyl cellulose (TAC) is 90% by mass, and hydroxypropyl cellulose (HPC) is 10% by mass. Example 13 is different from Example 1 in the combination of the water-insoluble polymer and the hydrophilizing agent. In Example 13, the critical wet surface tension was reduced by the combination of the water-insoluble polymer and the hydrophilizing agent, and the critical wet surface tension was 72 mN/m. In addition, the average through-hole diameter is 5.0 μm, and the fiber density difference is 0.90 as compared with Example 1.

### Example 14

In Example 14, triacetyl cellulose (TAC) was used as a water-insoluble polymer, and polyvinylpyrrolidone (PVP) was used as a hydrophilizing agent. For triacetyl cellulose (TAC), M-300 (trade name) manufactured by Eastman Chemical Company, Japan was used, and for polyvinylpyrrolidone (PVP), K-90 manufactured by Nippon Shokubai Co., Ltd. was used.

In Example 14, a nonwoven fabric was manufactured by an electrospinning method in the same manner as in Example 1 except that the average through-hole diameter, the void ratio, and the film thickness were changed as shown in Table 2 described later, and used as the cell separation filter. Triacetyl cellulose (TAC) is 90% by mass, and polyvinylpyrrolidone (PVP) is 10% by mass. In addition, the film thickness is 550 μm. In Example 14, the average through-hole diameter is 5.5 μm, the void ratio is 87%, and the film thickness is 500 μm as compared with Example 1.

### Example 15

In Example 15, triacetyl cellulose (TAC) was used as a water-insoluble polymer, and polyvinylpyrrolidone (PVP) was used as a hydrophilizing agent. For triacetyl cellulose (TAC), M-300 (trade name) manufactured by Eastman Chemical Company, Japan was used, and for polyvinylpyrrolidone (PVP), K-90 manufactured by Nippon Shokubai Co., Ltd. was used.

In Example 15, a nonwoven fabric was manufactured by an electrospinning method in the same manner as in Example 1 except that the average through-hole diameter, the void ratio, the film thickness, and the fiber density difference were changed as shown in Table 3 described later, and used as the cell separation filter. Triacetyl cellulose (TAC) is 90% by mass, and polyvinylpyrrolidone (PVP) is 10% by mass. In addition, in Example 15, the average through-hole diameter is 5.5 μm, the void ratio is 80%, the film thickness is 400 μm, and the fiber density difference is 0.89 as compared with Example 1.

### Example 16

In Example 16, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Example 16, a nonwoven fabric was manufactured by an electrospinning method in the same manner as in Example 1 except that the average through-hole diameter, the film thickness, and the fiber density difference were changed as shown in Table 3 described later, and used as the cell separation filter. Cellulose acetate propionate (CAP) is 90% by mass, and polyvinylpyrrolidone (PVP) is 10% by mass. In Example 16, the average through-hole diameter is 5.3 μm, the film thickness is 2,500 μm, and the fiber density difference is 0.76 as compared with Example 1.

### Example 17

In Example 17, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Example 17, a nonwoven fabric was manufactured by an electrospinning method in the same manner as in Example 1 except that the average through-hole diameter, the film thickness, and the fiber density difference were changed as shown in Table 3 described later, and used as the cell separation filter. Cellulose acetate propionate (CAP) is 90% by mass, and polyvinylpyrrolidone (PVP) is 10% by mass. In Example 17, the average through-hole diameter is 4.9 μm, the film thickness is 4,000 μm, and the fiber density difference is 0.70 as compared with Example 1.

### Example 18

In Example 18, polysulfone (PSU) was used as a water-insoluble polymer, and polyvinylpyrrolidone (PVP) was used as a hydrophilizing agent. For polysulfone (PSU), Udel (registered trade mark) P-3500 LCD MB manufactured by Solvay S.A. was used, and for polyvinylpyrrolidone (PVP), K-90 manufactured by Nippon Shokubai Co., Ltd. was used.

In Example 18, a nonwoven fabric was manufactured by an electrospinning method in the same manner as in Example 1 except that the average through-hole diameter, the void ratio, the critical wet surface tension, and the fiber density difference were changed as shown in Table 3 described later, and used as the cell separation filter. Polysulfone (PSU) is 90% by mass, and polyvinylpyrrolidone (PVP) is 10% by mass. The water-insoluble polymer of Example 18 is different from that of Example 1. In Example 18, the critical wet surface tension was reduced by the combination of the water-insoluble polymer and the hydrophilizing agent, and the critical wet surface tension was 72 mN/m. In addition, in Example 18, the average through-hole diameter is 3.5 μm, the void ratio is 90%, and the fiber density difference is 0.85 as compared with Example 1.

### Example 19

In Example 19, cellulose acetate propionate (CAP) was used as a water-insoluble polymer, and carboxymethyl cellulose (CMC) was used as a hydrophilizing agent. For cellulose acetate propionate (CAP), CAP-482-20 (trade name) manufactured by Eastman Chemical Company, Japan was used, and for carboxymethyl cellulose (CMC), product number 035-01337 manufactured by FUJIFILM Wako Pure Chemical Corporation was used.

In Example 19, a nonwoven fabric was manufactured by an electrospinning method in the same manner as in Example 1 except that the average through-hole diameter, the void ratio, and the fiber density difference were changed as shown in Table 3 described later, and used as the cell separation filter. Cellulose acetate propionate (CAP) is 90% by mass, and carboxymethyl cellulose (CMC) is 10% by mass. In Example 19, the average through-hole diameter is 3.3 μm, the void ratio is 94%, and the fiber density difference is 0.92 as compared with Example 1.

### Example 20

In Example 20, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Example 20, a nonwoven fabric was manufactured by an electrospinning method in the same manner as in Example 1 except that the average through-hole diameter, the void ratio, and the fiber density difference were changed as shown in Table 3 described later, and used as the cell separation filter. Cellulose acetate propionate (CAP) is 45% by mass, and polyvinylpyrrolidone (PVP) is 55% by mass. In Example 20, the average through-hole diameter is 3.6 μm, the void ratio is 95%, and the fiber density difference is 0.94 as compared with Example 1.

### Comparative Example 1

In Comparative Example 1, a nonwoven fabric having a film thickness of 500 μm was manufactured by a spun bonding method using polypropylene (PP). In Comparative Example 1, the average through-hole diameter is 2.9 μm, the void ratio is 80%, the critical wet surface tension is 30 mN/m, the film thickness is 500 μm, the fiber density difference is 0.99, and there is no fiber density gradient. That is, Comparative Example 1 is isotropic without anisotropy of fiber density.

For polypropylene (PP), WINTEC (registered trade mark) WSX02 manufactured by Japan Polypropylene Corporation was used.

### Comparative Example 2

In Comparative Example 2, a nonwoven fabric having a film thickness of 350 μm was manufactured by a melt blow method using polyethylene terephthalate (PET). In Comparative Example 2, the average through-hole diameter is 4.5 μm, the void ratio is 82%, the critical wet surface tension is 65 mN/m, the film thickness is 350 μm, the fiber density difference is 0.99, and there is no fiber density gradient. That is, Comparative Example 2 is isotropic without anisotropy of fiber density.

As the polyethylene terephthalate (PET), SA-1206 manufactured by UNITIKA Ltd. was used.

### Comparative Example 3

In Comparative Example 3, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Comparative Example 3, a nonwoven fabric was manufactured by an electrospinning method in the same manner as in Example 1 except that the average through-hole diameter, the film thickness, and the fiber density difference were changed as shown in Table 4 described later and there was no fiber density gradient, and used as the cell separation filter. Cellulose acetate propionate (CAP) is 90% by mass, and polyvinylpyrrolidone (PVP) is 10% by mass. In Comparative Example 3, the average through-hole diameter is 4.8 μm, the film thickness is 30 the fiber density difference is 0.99, and there is no fiber density gradient as compared with Example 1. That is, Comparative Example 3 is isotropic without anisotropy of fiber density.

### Comparative Example 4

In Comparative Example 4, only cellulose acetate propionate (CAP) was used without using a hydrophilizing agent. For cellulose acetate propionate (CAP), CAP-482-20 (trade name) manufactured by Eastman Chemical Company, Japan was used.

In Comparative Example 4, a nonwoven fabric was manufactured by an electrospinning method in the same manner as in Example 1 except that the average through-hole diameter, the void ratio, the critical wet surface tension, the film thickness, and the fiber density difference were changed as shown in Table 4 described later and there was no fiber density gradient, and used as the cell separation filter. In Comparative Example 4, the average through-hole diameter is 4.8 μm, the void ratio is 90%, the critical wet surface tension is 40 mN/m, the film thickness is 200 μm, the fiber density difference is 0.99, and there is no fiber density gradient as compared with Example 1. That is, Comparative Example 4 is isotropic without anisotropy of fiber density.

### Comparative Example 5

In Comparative Example 5, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Comparative Example 5, a nonwoven fabric was manufactured by an electrospinning method in the same manner as in Example 1 except that the average through-hole diameter and the fiber density difference were changed as shown in Table 4 described later, and used as the cell separation filter. Cellulose acetate propionate (CAP) is 90% by mass, and polyvinylpyrrolidone (PVP) is 10% by mass. In Comparative Example 5, the average through-hole diameter is 12.5 μm and the fiber density difference is 0.90 as compared with Example 1.

### Comparative Example 6

In Comparative Example 6, cellulose acetate propionate (CAP) was used as a water-insoluble polymer, and polyvinylpyrrolidone (PVP) was used as a hydrophilizing agent. For cellulose acetate propionate (CAP), CAP-482-20 (trade name) manufactured by Eastman Chemical Company, Japan was used, and for polyvinylpyrrolidone (PVP), K-90 manufactured by Nippon Shokubai Co., Ltd. was used.

In Comparative Example 6, a nonwoven fabric was manufactured by an electrospinning method in the same manner as in Example 1 except that the average through-hole diameter and the fiber density difference were changed as shown in Table 4 described later, and used as the cell separation filter. Cellulose acetate propionate (CAP) is 90% by mass, and polyvinylpyrrolidone (PVP) is 10% by mass. In Comparative Example 6, the average through-hole diameter is 0.9 μm and the fiber density difference is 0.90 as compared with Example 1.

### Comparative Example 7

In Comparative Example 7, triacetyl cellulose (TAC) was used as a water-insoluble polymer, and polyvinylpyrrolidone (PVP) was used as a hydrophilizing agent. For triacetyl cellulose (TAC), M-300 (trade name) manufactured by Eastman Chemical Company, Japan was used, and for polyvinylpyrrolidone (PVP), K-90 manufactured by Nippon Shokubai Co., Ltd. was used.

In Comparative Example 7, a nonwoven fabric was manufactured by an electrospinning method in the same manner as in Example 1 except that the average through-hole diameter, the void ratio, the film thickness, and the fiber density difference were changed as shown in Table 4 described later, and used as the cell separation filter. Triacetyl cellulose (TAC) is 90% by mass, and polyvinylpyrrolidone (PVP) is 10% by mass. In Comparative Example 7, the average through-hole diameter is 6.8 μm, the void ratio is 65%, the film thickness is 200 μm, and the fiber density difference is 0.92 as compared with Example 1.

TABLE 1

| | | Example 1 | Example 2 | Example 3 | Example 4 | Example 5 | Example 6 | Example 7 |
|---|---|---|---|---|---|---|---|---|
| Characteristics | Average through-hole | 3.1 | 5.0 | 5.0 | 5.0 | 2.1 | 9.7 | 9.7 |
| of cell | diameter (μm) | | | | | | | |
| separation filter | Void ratio (%) | 97 | 97 | 97 | 97 | 97 | 97 | 97 |
| | Wettability | 85 | 85 | 85 | 85 | 85 | 85 | 85 |
| | (CWST (mN/m)) | | | | | | | |
| | Film thickness (μm) | 800 | 800 | 800 | 250 | 800 | 800 | 800 |
| | Fiber density difference | 0.85 | 0.88 | 0.88 | 0.93 | 0.88 | 0.87 | 0.87 |
| | (L1/L10) | | | | | | | |
| | Fiber density gradient | Continuous | Continuous | Discontinuous | Discontinuous | Continuous | Continuous | Continuous |
| | Single layer/laminate | Single layer | Single layer | Single layer | Three layer | Single layer | Single layer | Single layer |
| | | | | | laminate | | | |
| | Material | CAP/90% | CAP/90% | CAP/90% | CAP/90% | CAP/90% | CAP/90% | CAP/90% |
| | Hydrophilizing agent | PVP/10% | PVP/10% | PVP/10% | PVP/10% | PVP/10% | PVP/10% | PVP/10% |
| | Manufacturing method | Electro- | Electro- | Electro- | Electro- | Electro- | Electro- | Electro- |
| | | spinning | spinning | spinning | spinning | spinning | spinning | spinning |
| Blood | Secondary filter | Absent | Absent | Absent | Absent | Absent | Absent | PSU |
| filtration test | Degree of hemolysis | 0.2% | 0.1% | 1.5% | 1.8% | 3.2% | 2.5% | 0.8% |
| | | A | A | B | B | B | B | A |
| | Processing pressure | A | A | B | A | A | A | A |
| | Component matching | A | A | A | B | A | A | A |
| | rate after filtration | | | | | | | |

TABLE 2

| | | Example 8 | Example 9 | Example 10 | Example 11 | Example 12 | Example 13 | Example 14 |
|---|---|---|---|---|---|---|---|---|
| Characteristics | Average through-hole | 44 | 4.1 | 3.8 | 7.2 | 3.3 | 5.0 | 5.5 |
| of cell | diameter (μm) | | | | | | | |
| separation filter | Void ratio (%) | 96 | 96 | 98 | 95 | 97 | 97 | 87 |
| | Wettability | 85 | 85 | 85 | 85 | 72 | 72 | 85 |
| | (CWST (mN/m)) | | | | | | | |
| | Film thickness (μm) | 500 | 500 | 150 | 200 | 800 | 800 | 550 |
| | Fiber density difference | 0.90 | 0.84 | 0.94 | 0.94 | 0.90 | 0.90 | 0.85 |
| | (L1/L10) | | | | | | | |
| | Fiber density gradient | Continuous | Continuous | Continuous | Continuous | Continuous | Continuous | Continuous |
| | Single layer/laminate | Single layer | Single layer | Single layer | Single layer | Single layer | Single layer | Single layer |
| | Material | TAC/90% | DAC/90% | TAC/90% | TAC/90% | CAP/92.5% | TAC/90% | TAC/90% |
| | Hydrophilizing agent | PVP/10% | PVP/10% | PVP/10% | PVP/10% | PVP/7.5% | HPC/10% | PVP/10% |
| | Manufacturing method | Electro- | Electro- | Electro- | Electro- | Electro- | Electro- | Electro- |
| | | spinning | spinning | spinning | spinning | spinning | spinning | spinning |
| Blood | Secondary filter | Absent | Absent | Absent | Absent | Absent | Absent | Absent |
| filtration test | Degree of hemolysis | 0.2% | 0.2% | 2.5% | 1.2% | 1.1% | 1.6% | 0.9% |
| | | A | A | B | B | B | B | B |
| | Processing pressure | A | A | A | A | A | A | A |
| | Component matching | A | A | A | A | A | A | A |
| | rate after filtration | | | | | | | |

TABLE 3

| | | Example 15 | Example 16 | Example 17 | Example 18 | Example 19 | Example 20 |
|---|---|---|---|---|---|---|---|
| Characteristics | Average through-hole | 5.5 | 5.3 | 4.9 | 3.5 | 3.3 | 3.6 |
| of cell | diameter (μm) | | | | | | |
| separation filter | Void ratio (%) | 80 | 97 | 97 | 90 | 94 | 95 |
| | Wettability | 85 | 85 | 85 | 72 | 85 | 85 |
| | (CWST (mN/m)) | | | | | | |
| | Film thickness (μm) | 400 | 2500 | 4000 | 800 | 800 | 800 |
| | Fiber density difference | 0.89 | 0.76 | 0.70 | 0.85 | 0.92 | 0.94 |
| | (L1/L10) | | | | | | |
| | Fiber density gradient | Continuous | Continuous | Continuous | Continuous | Continuous | Continuous |
| | Single layer/laminate | Single layer | Single layer | Single layer | Single layer | Single layer | Single layer |
| | Material | TAC/90% | CAP/90% | CAP/90% | PSU/90% | CAP/90% | CAP/45% |
| | Hydrophilizing agent | PVP/10% | PVP/10% | PVP/10% | PVP/10% | CMC/10% | PVP/10% |
| | Manufacturing method | Electro- | Electro- | Electro- | Electro- | Electro- | Electro- |
| | | spinning | spinning | spinning | spinning | spinning | spinning |
| Blood | Secondary filter | Absent | Absent | Absent | Absent | Absent | Absent |
| filtration test | Degree of hemolysis | 1.7% | 1.9% | 3.3% | 0.8% | 1.2% | 1.5% |
| | | B | B | B | A | B | B |
| | Processing pressure | B | A | B | A | B | B |
| | Component matching | A | A | B | B | B | B |
| | rate after filtration | | | | | | |

TABLE 4

| | | Comparative | Comparative | Comparative | Comparative | Comparative | Comparative | Comparative |
|---|---|---|---|---|---|---|---|---|
| | | Example 1 | Example 2 | Example 3 | Example 4 | Example 5 | Example 6 | Example 7 |
| Characteristics | Average through-hole | 2.9 | 4.5 | 4.8 | 4.8 | 12.5 | 0.9 | 6.8 |
| of cell | diameter (μm) | | | | | | | |
| separation filter | Void ratio (%) | 80 | 82 | 97 | 90 | 97 | 97 | 65 |
| | Wettability | 30 | 65 | 85 | 40 | 85 | 85 | 85 |
| | (CWST (mN/m)) | | | | | | | |
| | Film thickness (μm) | 500 | 350 | 30 | 200 | 800 | 800 | 200 |
| | Fiber density difference | 0.99 | 0.99 | 0.99 | 0.99 | 0.90 | 0.90 | 0.92 |
| | (L1/L10) | | | | | | | |
| | Fiber density gradient | Absent | Absent | Absent | Absent | Continuous | Continuous | Continuous |
| | Single layer/laminate | Single layer | Single layer | Single layer | Single layer | Single layer | Single layer | Single layer |
| | Material | PP | PET | CAP/90% | CAP | CAP/90% | CAP/90% | TAC/90% |
| | Hydrophilizing agent | — | — | PVP/I0% | — | PVP/10% | PVP/10% | PVP/10% |
| | Manufacturing method | Spun | Melt blow | Electro- | Electro- | Electro- | Electro- | Electro- |
| | | bonding | | spinning | spinning | spinning | spinning | spinning |
| Blood | Secondary filter | Absent | Absent | Absent | Absent | Absent | Absent | Absent |
| filtration test | Degree of hemolysis | 25.0% | 18.9% | 12.5% | 11.0% | 15.5% | 13.2% | 7.5% |
| | | D | D | D | D | D | D | C |
| | Processing pressure | D | C | C | C | C | C | C |
| | Component matching | D | D | C | C | D | D | C |
| | rate after filtration | | | | | | | |

As shown in Table 1 to Table 4, Examples 1 to 20 were excellent in all evaluations of the degree of hemolysis, the processing pressure, and the component matching rate after filtration as compared with Comparative Examples 1 to 7.

In Comparative Example 1, the composition and the manufacturing method for a cell separation filter are different, there is no hydrophilizing agent, the critical wet surface tension (CWST) is low, and the fiber density difference is small. In Comparative Example 1, all the evaluations of the degree of hemolysis, the processing pressure, and the component matching rate after filtration are bad.

In Comparative Example 2, the composition and the manufacturing method for a cell separation filter are different, there is no hydrophilizing agent, the critical wet surface tension (CWST) is low, and the fiber density difference is small. In Comparative Example 2, all the evaluations of the degree of hemolysis and the component matching rate after filtration were band, but the processing pressure was slightly good as compared with those of Comparative Example 1.

In Comparative Example 3, the film thickness is thin, and the fiber density difference is small. In Comparative Example 3, all the evaluations of the degree of hemolysis, the processing pressure, and the component matching rate after filtration were bad, but the processing pressure and the component matching rate after filtration were slightly good as compared with those of Comparative Example 1.

In Comparative Example 4, there is no hydrophilizing agent, the critical wet surface tension (CWST) is low, and the fiber density difference is small. In Comparative Example 4, all the evaluations of the degree of hemolysis, the processing pressure, and the component matching rate after filtration were bad, but the processing pressure and the component matching rate after filtration were slightly good as compared with those of Comparative Example 1.

In Comparative Example 5, the average through-hole diameter was large and all the evaluations of the degree of hemolysis, the processing pressure, and the component matching rate after filtration were bad, but the processing pressure was slightly good as compared with that of Comparative Example 1.

In Comparative Example 6, the average through-hole diameter was small and all the evaluations of the degree of hemolysis, the processing pressure, and the component matching rate after filtration were bad, but the processing pressure was slightly good as compared with that of Comparative Example 1.

In Comparative Example 7, the void ratio was low and all the evaluations of the degree of hemolysis, the processing pressure, and the component matching rate after filtration were bad, but the degree of hemolysis, the processing pressure, and the component matching rate after filtration were slightly good as compared with those of Comparative Example 1.

From Example 1, Example 5, and Example 6, it can be seen that there is an average through-hole diameter that is excellent in the degree of hemolysis. Further, from Example 6 and Example 7, it can be seen that the degree of hemolysis is excellent in a case where a porous body which is the secondary filter is provided.

From Example 2, Example 3, and Example 4, it can be seen that the degree of hemolysis and the processing pressure are excellent in a case where the fiber density gradient is continuous.

From Example 1 and Example 10, it can be seen that the degree of hemolysis is excellent in a case where the film thickness is thicker.

From Example 1, Example 12, and Example 13, it can be seen that in a case where the critical wet surface tension is high, the degree of hemolysis is excellent.

From Example 2, Example 14, and Example 15, it can be seen that Example 2 having a void ratio of 97% is excellent in the degree of hemolysis as compared with Example 14 having a void ratio of 87%, and further excellent in the degree of hemolysis and the processing pressure as compared with Example 15 having a void ratio of 80%.

From Example 2, Example 16, and Example 17, it can be seen that Example 2 having a film thickness of 800 μm is excellent in the degree of hemolysis as compared with Example 16 having a film thickness of 2,500 μm, and further excellent in the degree of hemolysis, the processing pressure, and the component matching rate as compared with Example 17 having a film thickness of 4,000 μm.

From Example 1 and Example 19, it can be seen that the hydrophilizing agent is preferably polyvinylpyrrolidone (PVP). Further, from Example 1 and Example 20, it can be seen that the content of the hydrophilizing agent is preferably 50% by mass or less.

The hydrophilizing agent is preferably polyvinylpyrrolidone (PVP) from the viewpoint that polyvinylpyrrolidone has high compatibility with a water-insoluble polymer as compared with other materials, has a result of high hydrophilicity, and the critical wet surface tension (CWST) of the nonwoven fabric becomes high and the viewpoint of evaluation of the degree of hemolysis, the processing pressure, and the component matching rate after filtration.

In a case where the content of the hydrophilizing agent is more than 50% by mass, the strength of the fibers forming the nonwoven fabric is decreased, the shape is easily changed by filtration, whereby the processing pressure is increased. As a result, the content thereof is preferably 50% by mass or less.

### EXPLANATION OF REFERENCES

10: cell separation filter
12: nonwoven fabric
12a: front surface
12b: back surface
14: porous body
20: filtering device
22: case
22a: inside
22b: bottom part
22c: opening portion
22d: outer surface
24: connecting pipe
26: collection unit
27: supply pipe
28: pressurizing part
28a: gasket
28b: plunger
30: filtration system
32: supply unit
34: pipe
36: control unit
50: pressure curve
52: pressure curve
100: conventional nonwoven fabric
Dt: film thickness direction
h: film thickness

What is claimed is:

1. A cell separation filter comprising a nonwoven fabric that is formed of fibers containing a water-insoluble polymer and a hydrophilizing agent and has a fiber density difference in a film thickness direction,
wherein the nonwoven fabric has an average through-hole diameter of 2.0 μm or more and less than 10.0 μm, a void ratio of 75% or more and 98% or less, a film thickness of 100 μm or more, and a critical wet surface tension of 72 mN/m or more.

2. The cell separation filter according to claim 1,
wherein the hydrophilizing agent is at least one of polyvinylpyrrolidone, polyethylene glycol, carboxymethyl cellulose, or hydroxypropyl cellulose.

3. The cell separation filter according to claim 1,
wherein the nonwoven fabric has a film thickness of 200 μm or more and 2,000 μm or less.

4. The cell separation filter according to claim 1,
wherein the critical wet surface tension is 85 mN/m or more.

5. The cell separation filter according to claim 1,
wherein the water-insoluble polymer is any one of polyethylene, polypropylene, polyester, polysulfone, polyethersulfone, polycarbonate, polystyrene, a cellulose derivative, an ethylene vinyl alcohol polymer, polyvinyl chloride, polylactic acid, polyurethane, polyphenylene sulfide, polyamide, polyimide, polyvinylidene fluoride, polytetrafluoroethylene, or an acrylic resin, or a mixture thereof.

6. The cell separation filter according to claim 1,
wherein the water-insoluble polymer is formed of a cellulose derivative.

7. The cell separation filter according to claim 1,
wherein a content of the hydrophilizing agent with respect to a total mass of the fibers of the nonwoven fabric is 1% to 50% by mass.

8. The cell separation filter according to claim 1,
wherein a fiber density of the nonwoven fabric continuously changes in the film thickness direction.

9. A filtering device comprising the cell separation filter according to claim 1,
wherein the cell separation filter is arranged so that a filtration target passes through the cell separation filter from a low fiber density side to a high fiber density side in a film thickness direction.

10. A filtering device comprising:
the cell separation filter according to claim 1; and
a porous body having an average through-hole diameter of 0.2 μm or more and 1.5 μm or less and a void ratio of 60% or more and 95% or less,
wherein the cell separation filter and the porous body are arranged so that a filtration target passes through the cell separation filter and the porous body in this order.

11. The filtering device according to claim 10,
wherein the cell separation filter is arranged so that a filtration target passes through the cell separation filter from a low fiber density side to a high fiber density side in a film thickness direction.

12. A manufacturing method for the cell separation filter according to claim 1,
wherein the cell separation filter is manufactured by using an electrospinning method.

13. The cell separation filter according to claim 2,
wherein the nonwoven fabric has a film thickness of 200 μm or more and 2,000 μm or less.

14. The cell separation filter according to claim 2,
wherein the critical wet surface tension is 85 mN/m or more.

15. The cell separation filter according to claim 2,
wherein the water-insoluble polymer is any one of polyethylene, polypropylene, polyester, polysulfone, polyethersulfone, polycarbonate, polystyrene, a cellulose derivative, an ethylene vinyl alcohol polymer, polyvinyl chloride, polylactic acid, polyurethane, polyphenylene sulfide, polyamide, polyimide, polyvinylidene fluoride, polytetrafluoroethylene, or an acrylic resin, or a mixture thereof.

16. The cell separation filter according to claim 2,
wherein the water-insoluble polymer is formed of a cellulose derivative.

17. The cell separation filter according to claim 2,
wherein a content of the hydrophilizing agent with respect to a total mass of the fibers of the nonwoven fabric is 1% to 50% by mass.

18. The cell separation filter according to claim 2,
wherein a fiber density of the nonwoven fabric continuously changes in the film thickness direction.

19. A filtering device comprising the cell separation filter according to claim 2,
wherein the cell separation filter is arranged so that a filtration target passes through the cell separation filter from a low fiber density side to a high fiber density side in a film thickness direction.

20. A filtering device comprising:
the cell separation filter according to claim 2; and
a porous body having an average through-hole diameter of 0.2 μm or more and 1.5 μm or less and a void ratio of 60% or more and 95% or less,
wherein the cell separation filter and the porous body are arranged so that a filtration target passes through the cell separation filter and the porous body in this order.
